(19) United States
(12) Patent Application Publication
(10) Pub. No.: US 2020/0297886 A1
(43) Pub. Date: Sep. 24, 2020

(54) DEODORANT-CONTAINING WORKING FLUID, METHOD FOR MANUFACTURING DEODORIZING PRODUCT, DEODORIZING FILTER MEDIUM, DEODORIZING FILTER UNIT, AND DEODORIZING DEVICE

(71) Applicant: TOAGOSEI CO., LTD., Minato-ku (JP)

(72) Inventors: Yuuki UESUGI, Nagoya-shi (JP); Yoshinao YAMADA, Nagoya-shi (JP)

(73) Assignee: TOAGOSEI CO., LTD., Minato-ku (JP)

(21) Appl. No.: 16/763,830

(22) PCT Filed: Nov. 5, 2018
(86) PCT No.: PCT/JP2018/040990

§ 371 Date: May 13, 2020

(30) Foreign Application Priority Data
Nov. 16, 2017 (JP) 2017-221313

(51) Int. Cl. A61L 9/014; B01J 20/22; B01D 53/02; A61L 9/16; B01J 20/28; B01J 20/32

(52) U.S. Cl. CPC A61L 9/014; B01J 20/22; B01J 20/3246; A61L 9/16; B01J 20/28023; B01D 53/02

(57) ABSTRACT

The present deodorant-containing processing liquid includes an acid salt of an aminoguanidine, an inorganic carrier capable of carrying the acid salt of the aminoguanidine, a dispersant, and water, a content of the acid salt of the aminoguanidine is 15 parts or more by mass based on 100 parts by mass of a content of the inorganic carrier, and the dispersant is at least one selected from a group consisting of an anionic surfactant and a nonionic surfactant.

### FIELD OF THE INVENTION

The present invention relates to a deodorant-containing processing liquid, a method for producing a deodorizing product, a deodorizing filter medium, a deodorizing filter unit, and a deodorizing apparatus.

### BACKGROUND ART

Depending on a living environment or a working environment, various gaseous pollutants such as bad odors and harmful gases are present in air, and therefore, attention has been paid to the removal of these gaseous pollutants to establish a comfortable environment. For example, aldehyde-based gases are contained in various odors such as cigarette smoke, body odor (e.g., sweat odor, breath odor), pet odor, mold odor, paint odor, and printing odor, and the influences of the aldehyde-based gases on human health have been pointed out. In recent years, deodorizing filter media, deodorizing filters, and the like have been proposed to clarify air containing aldehyde-based gases in rooms, cars, etc.

As conventional deodorizing filter media for aldehyde-based gases, the following products are known which are obtained by adhering a chemical adsorption-type deodorant to nonwoven fabrics, paper, and the like. In the following literatures, the term “deodorizing filter (or deodorant filter)” is used instead of the deodorizing filter medium.

Patent Literature 1 discloses a deodorizing filter comprising a deodorant containing a catalyst obtained by allowing manganese oxide to carry a ruthenium compound or a phosphoric acid salt compound.

Patent Literature 2 discloses a deodorizing filter containing a particle carrying a hydroxyamine compound such as 2-amino-2-hydroxymethyl-1,3-propanediol and a porous polymer particle obtained by copolymerizing monomer components including a cross-linkable vinyl monomer and a vinyl monomer having a nitrogen-containing aromatic ring.

Patent Literature 3 discloses a deodorizing filter obtained by allowing an activated carbon-containing paper to carry a cyclic saturated amine and a basic compound.

When a deodorizing product is produced, a deodorant is used according to its properties. In general, a method is used in which a dispersion liquid containing a deodorant, an adhesive, and a medium, that is, a deodorant-containing processing liquid is applied onto a surface of a base and a resulting coating film is dried. The deodorant-containing processing liquid conventionally contains a dispersant from a viewpoint of the stability of the liquid. Patent Literature 4 discloses a deodorant-dispersed liquid containing a deodorant obtained by allowing aluminum silicate to carry an acid salt of aminoguanidine, a dispersant, a preservative, an antifoaming agent, a thickener, a binder, and water, and the deodorant-dispersed liquid is a suspension liquid (deodorant-containing processing liquid) containing, as the dispersant, an alkylammonium salt of a block copolymer containing a phosphoric acid group (amphoteric surfactant) or polyoxyethylene nonylphenyl ether (nonionic surfactant). This suspension liquid is used to produce a deodorizing fiber or a deodorizing carpet.

### PRIOR ART LITERATURE

### Patent Literature

Patent Literature 1: JP-A 2008-104845

Patent Literature 2: JP-A 2012-120637

Patent Literature 3: JP-A 2016-154640

Patent Literature 4: WO 2007/88879

### SUMMARY OF THE INVENTION

### Problems that the Invention is to Solve

Deodorizing performance of a deodorizing product obtained by impregnating a base with a deodorant generally depends on an impregnation amount of the deodorant. Therefore, when a deodorizing product is produced using a deodorant-containing processing liquid, it is necessary to use a deodorant-containing processing liquid containing a deodorant at high concentration or to repeatedly perform application and drying of a deodorant-containing processing liquid containing a deodorant at low concentration in order to impregnate a base with a larger amount of the deodorant. However, when a deodorant-containing processing liquid containing a deodorant at high concentration is used, defects are caused, such as generation of an aggregate and thickening of the liquid. On the other hand, the latter approach in which a deodorant-containing processing liquid containing a deodorant at low concentration is repeatedly applied and dried is not advantageous in terms of production costs.

### Means for Solving the Problems

In light of the above problem of the prior art, the present inventors have studied a deodorant-containing processing liquid for producing a deodorizing product by impregnating a base with a deodorant to complete the present invention. More specifically, the present inventors have found that a deodorant-containing processing liquid including an acid salt of aminoguanidine, an inorganic carrier carrying the acid salt of aminoguanidine in a predetermined amount or more with respect to an amount of the inorganic carrier, at least one selected from an anionic surfactant and a nonionic surfactant as a dispersant, and a dispersion medium has excellent storage stability and can be used to provide a deodorizing product that has high deodorizing performance.

The present invention is as follows.

1. A deodorant-containing processing liquid comprising an acid salt of an aminoguanidine, an inorganic carrier capable of carrying the acid salt of the aminoguanidine, a dispersant, and a dispersion medium, characterized in that a content of the acid salt of the aminoguanidine is 15 parts or more by mass based on 100 parts by mass of a content of the inorganic carrier, and that the dispersant is at least one selected from a group consisting of an anionic surfactant and a nonionic surfactant.
2. The deodorant-containing processing liquid according to 1 above, wherein the acid salt of the aminoguanidine is carried by the inorganic carrier.
3. The deodorant-containing processing liquid according to 1 or 2 above, wherein a content ratio of the inorganic carrier is in a range from 0.1% to 50% by mass based on the deodorant-containing processing liquid.
4. A method for producing a deodorizing product, characterized by comprising the steps of: forming a coating film on a surface of a base using the deodorant-containing processing liquid according to any one of 1 to 3 above and the base; and drying the coating film.
5. A deodorizing filter medium comprising a sheet-shaped base comprising a fiber and a deodorant-containing part joined to a surface of the fiber, the deodorizing filter medium having an air permeable from one surface side to other surface side, characterized in that the deodorant-containing part comprises an acid salt of an aminoguanidine, an inorganic carrier carrying the acid salt of the aminoguanidine, an adhesive resin, and a surfactant, that a content of the acid salt of the aminoguanidine is 15 parts or more by mass based on 100 parts by mass of a content of the inorganic carrier, and that the surfactant is at least one selected from a group consisting of an anionic surfactant and a nonionic surfactant.
6. The deodorizing filter medium according to 5 above, wherein a content ratio of the acid salt of the aminoguanidine is in a range from 1% to 50% by mass based on the base.
7. The deodorizing filter medium according to 5 or 6 above, wherein an air permeability of the deodorizing filter medium is in a range from 10 to 500 cm³/(cm²·s).
8. A deodorizing filter unit characterized by comprising the deodorizing filter medium according to any one of 5 to 7 above and a carrying member that carries the deodorizing filter medium.
9. A deodorizing apparatus characterized by comprising the deodorizing filter medium according to any one of 5 to 7 above.

### Effects of the Invention

The deodorant-containing processing liquid according to an embodiment of the present invention is excellent in stability, easy to handle, and suitable for producing a deodorizing product for aldehyde-based gases whose impregnation amount of a deodorant is high.

The production method of a deodorizing product according to the embodiment of the present invention makes it possible to provide a deodorizing product for aldehyde-based gases which has high deodorizing performance.

The deodorizing filter medium according to the embodiment of the present invention has high deodorizing performance against aldehyde-based gases. Therefore, the deodorizing filter unit and the deodorizing apparatus having the deodorizing filter medium according to the embodiment of the present invention also have high deodorizing performance against aldehyde-based gases.

### BRIEF DESCRIPTION OF THE DRAWINGS

FIG. 1 is a schematic diagram showing an example of the cross-sectional structure of a deodorizing filter medium according to an embodiment of the present invention; and

FIG. 2 is a schematic diagram for explaining an aeration deodorant test for deodorizing filter medium in Examples.

### EMBODIMENT FOR CARRYING OUT THE INVENTION

The deodorant-containing processing liquid according to an embodiment of the present invention is a composition including an acid salt of aminoguanidine, an inorganic carrier capable of carrying the acid salt of aminoguanidine, an adhesive resin, a dispersant, and water as a dispersion medium. The acid salt of aminoguanidine and the inorganic carrier function as a deodorant for aldehyde-based gases. The deodorant-containing processing liquid according to the embodiment of the present invention, if necessary, may further include other deodorant (described later) that chemically adsorbs aldehyde-based gases or other odorous gases and/or an additive.

The deodorant-containing processing liquid according to the embodiment of the present invention is intended to produce a deodorizing product by applying it onto a base having a predetermined shape and then drying a resulting coating film so that a deodorant containing the acid salt of aminoguanidine and the inorganic carrier is adhered to the base with the adhesive resin. It is to be noted that the term “aldehyde-based gases” used in the embodiment of the present invention refers to gases derived from compounds having a —CHO group. Specific examples thereof include gases derived from formaldehyde, acetaldehyde, propanal, butanal, nonenal, or the like.

The acid salt of aminoguanidine may be a hydrochloride, a sulfate, a carbonate, a nitrate, or the like of a monoaminoguanidine, a diaminoguanidine, or a triaminoguanidine.

Examples of the monoaminoguanidine salt include aminoguanidine hydrochloride, aminoguanidine sulfate, aminoguanidine bicarbonate, aminoguanidine nitrate, and the like.

Examples of the diaminoguanidine salt include diaminoguanidine hydrochloride, diaminoguanidine sulfate, diaminoguanidine nitrate, and the like.

Examples of the triaminoguanidine salt include triaminoguanidine hydrochloride, triaminoguanidine nitrate, and the like.

The acid salt of aminoguanidine contained in the deodorant-containing processing liquid of the present embodiment may be only one type, or two or more types.

Aminoguanidine hydrochloride and aminoguanidine sulfate are preferable as the acid salt of aminoguanidine.

The inorganic carrier is not particularly limited as long as the inorganic carrier docs not react with water and maintains its properties in the deodorant-containing processing liquid. Examples of a material constituting the inorganic carrier include a silicate compound, a tetravalent metal phosphate compound, a zeolite, a silica gel, and the like.

Among these, a silicate compound and a silica gel are particularly preferable.

The silicate compound is preferably an aluminum silicate or a magnesium silicate.

The aluminum silicate is preferably a compound represented by the following general formula (1), and the magnesium silicate is preferably a compound represented by the following general formula (2).

Al₂O₃.mSiO₂.nH₂O (1)

(In the formula, m is an integer of 6 or more, and n is an integer of 1 or more.)

MgO.qSiO₂.nH₂O (2)

(In the formula, q is an integer of 1 or more, and n is an integer of 0.1 or more.)

In the general formula (1) representing the aluminum silicate, m is preferably 6 to 50, and more preferably 8 to 15. Additionally, n is preferably 1 to 20, and more preferably 3 to 15.

In the general formula (2) representing the magnesium silicate, q is preferably 1 to 20, and more preferably 3 to 15. Additionally, n is preferably 0.1 to 20, and more preferably 1 to 8.

The silicate compound is particularly preferably an aluminum silicate.

Examples of the tetravalent metal phosphate compound include zirconium phosphate, titanium phosphate, tin phosphate, and the like. These compounds may either be crystalline or amorphous.

The zeolite may have a structure of type A, type X, type Y, type α, type β, or ZSM-5. The zeolites of these types may either be natural products or synthetic products.

The properties and size of the inorganic carrier are not particularly limited. From a viewpoint of a deodorizing effect on aldehyde-based gases, a BET specific surface area is preferably 10 m²/g or more, and more preferably 50 m²/g or more, and an average particle diameter is preferably in a range from 0.01 to 50 μm, and more preferably from 0.02 to 20 μm. If the BET specific surface area exceeds 2,000 m²/g, the pore diameter of the inorganic carrier is smaller than the particle diameter of the acid salt of aminoguanidine, and therefore it is difficult for the inorganic carrier to carry the acid salt of aminoguanidine.

The deodorant-containing processing liquid according to the embodiment of the present invention includes at least a complex obtained by allowing the inorganic carrier to carry the acid salt of aminoguanidine from a viewpoint of deodorizing performance of a resulting deodorizing product.

From a viewpoint of deodorizing effect of the complex, a content of the acid salt of aminoguanidine in the complex obtained by allowing the inorganic carrier to carry the acid salt of aminoguanidine is 15 parts or more by mass, preferably in a range from 15 to 200 parts by mass, more preferably from 20 to 160 parts by mass, and further preferably from 25 to 100 parts by mass based on 100 parts by mass of the inorganic carrier. If the content of the acid salt of aminoguanidine is less than 15 parts by mass against the inorganic carrier, a sufficient deodorizing effect cannot be obtained, in which case the base needs to be processed two or more times by applying the deodorant-containing processing liquid thereonto to obtain a desired deodorizing effect. On the other hand, if the content of the acid salt of aminoguanidine exceeds 200 parts by mass against the inorganic carrier, there is a case where deodorizing efficiency is reduced because the amount of aminoguanidine with respect to the surface area of the inorganic carrier is excessively high

Further, even when the complex is used in combination with a deodorant for gases other than aldehyde-based gases, such as sulfur-based gases, basic gases, and organic acid gases, deodorizing performance against aldehyde-based gases can sufficiently be delivered while deodorizing performance against sulfur-based gases, basic gases, organic acid gases, or the like is maintained.

From a viewpoint of deodorizing performance of a resulting deodorizing product, a content of the inorganic carrier contained in the deodorant-containing processing liquid of the embodiment of the present invention is preferably in a range from 0.1% to 50% by mass, more preferably from 1% to 40% by mass, and further preferably from 5% to 30% by mass based on the deodorant-containing processing liquid. If the content of the inorganic carrier is less than 0.1% by mass, deodorizing performance cannot sufficiently be achieved, and therefore the base needs to be processed two or more times to obtain a desired deodorizing effect. On the other hand, if the content of the inorganic carrier exceeds 50% by mass, there is a case where the deodorant-containing processing liquid is not suitable for processing due to its too high a viscosity or a precipitate is formed due to the aggregation of the inorganic carrier.

The complex may be obtained by a conventionally publicly known method. For example, the complex may be obtained by a method in which a solution (aqueous solution or alcohol solution) of the acid salt of aminoguanidine is dropped or sprayed onto a powder of the inorganic carrier with stirring, and then a medium is removed by heating.

The adhesive resin is a component to adhere at least the acid salt of aminoguanidine and the inorganic carrier to a base for producing a deodorizing product. The adhesive resin contained in the deodorant-containing processing liquid according to the embodiment of the present invention may be only one type, or two or more types.

The adhesive resin may either be a water-soluble resin or a water-insoluble resin. Example thereof includes an ethylene-vinyl acetate copolymer or a modified product thereof (e.g., an acid-modified product), an ethylene-vinyl chloride copolymer or a modified product thereof, a vinyl chloride-vinyl acetate copolymer, polyvinyl acetate, polyvinyl chloride, a modified olefin resin (e.g., chlorinated polyolefin), a polyester resin, an acrylic resin, a urethane resin or a modified product thereof, a styrene-butadiene copolymer, a styrene-isoprene copolymer, a styrene-butadiene-styrene block copolymer, a styrene-ethylene-butylene-styrene block copolymer, a styrene-ethylene-propylene-styrene block copolymer, a hydrogenated styrene-bu adiene-styrene block copolymer, a hydrogenated styrene-ethylene-butylene-styrene block copolymer, a hydrogenated styrene-ethylene-propylene-styrene block copolymer, and the like. Among these, an ethylene-vinyl acetate copolymer or a modified product thereof (e.g., an acid-modified product), an ethylene-vinyl chloride copolymer or a modified product, a vinyl chloride-vinyl acetatecopolymer, a poly vinyl acetate, a poly vinyl chloride, a modified polyolefin (e.g., chlorinated polyolefin), a polyester resin, an acrylic resin, and a urethane resin or a modified product thereof are preferable.

When the adhesive resin is a water-insoluble resin, the adhesive resin may be in the form of particles, lines, or the like in the deodorant-containing processing liquid according to the embodiment of the present invention.

A content of the adhesive resin contained in the deodorant-containing processing liquid according to the embodiment of the present invention is preferably in a range from 10 to 300 parts by mass, more preferably from 15 to 200 parts by mass, and further preferably from 20 to 150 parts by mass based on 100 parts by mass of a total amount of the acid salt of aminoguanidine and the inorganic carrier or a total amount of the acid salt of aminoguanidine, the inorganic carrier, and other deodorant (described later) that is optionally contained.

The dispersant is at least one selected from an anionic surfactant and a nonionic surfactant, and imparts stability to the deodorant-containing processing liquid according to the embodiment of the present invention when, as described above, the content of the acid salt of aminoguanidine is 15 parts or more by mass based on 100 parts by mass of the inorganic carrier. The dispersant contained in the deodorant-containing processing liquid according to the embodiment of the present invention may be either one or both of the anionic surfactant and the nonionic surfactant.

Examples of the anionic surfactant include a carboxylic acid-based anionic surfactant, a sulfonic acid-based anionic surfactant, a sulfuric acid-based anionic surfactant, a phosphoric acid-based anionic surfactant, and the like. Among these, a carboxylic acid-based anionic surfactant is preferable. The carboxylic acid-based anionic surfactant contained in the dispersant may be only one type, or two or more types.

Hereinafter, specific examples of the anionic surfactant will be described, and the term “salt” refers to an alkali metal salt (e.g., a lithium salt, a sodium salt, a potassium salt), an alkaline earth metal salt (e.g., a magnesium salt, a calcium salt), an ammonium salt, or an amine salt (e.g., a monoethanolamine salt, a diethanolamine salt, a triethanolamine salt, a monoisopropanolamine salt).

Examples of the carboxylic acid-based anionic surfactant include a polycarboxylic acid salt, an aliphatic carboxylic acid salt, an alkyl ether carboxylic acid salt, an alkenyl succinic acid salt, an N-acylamino acid salt, an amide ether carboxylic acid salt, an acyl lactic acid salt, and the like. Among these, a a polycarboxylic acid salt is preferable.

Examples of the polycarboxylic acid salt include salts of homopolymers or copolymers containing a structural unit derived from acrylic acid, methacrylic acid, maleic acid, fumaric acid, itaconic acid, maleic anhydride, and the like. Specifically, salts of polyacrylic acid, polymethacrylic acid, polymaleic acid, polymaleic anhydride, a maleic acid-isobutylene copolymer, a maleic anhydride-isobutylene copolymer, a maleic acid-diisobutylene copolymer, a maleic anhydride-diisobutylene copolymer, an acrylic acid-itaconic acid copolymer, a methacrylic acid-itaconic acid copolymer, a maleic acid-styrene copolymer, a maleic anhydride-styrene copolymer, an acrylic acid-methacrylic acid copolymer, an acrylic acid-acrylic acid methyl ester copolymer, an acrylic acid-vinyl acetate copolymer, an acrylic acid-maleic acid copolymer, an acrylic acid-maleic anhydride copolymer, and the like can be used.

Examples of the aliphatic carboxylic acid salt include an aliphatic carboxylic acid salt containing an alkyl group having 8 to 20 carbon atoms, such as a caproic acid salt, a caprylic acid salt, a capric acid salt, a lauric acid salt, a myristic acid salt, a palmitic acid salt, a stearic acid salt, and an oleic acid salt.

Examples of the alkyl ether carboxylic acid salt include a (poly)oxyethylene alkyl ether acetic acid salt, a (poly)oxyethylene alkyl ether propionic acid salt, and the like. Specifically, an alkyl ether carboxylic acid salt containing an alkylene group having 14 to 60 carbon atoms, such as an oxyethylene oleyl ether acetic acid salt, a polyoxyethylene lauryl ether acetic acid salt, a polyoxyethylene stearyl ether acetic acid salt, a polyoxyethylene hexylphenyl ether acetic acid salt, a polyoxyethylene tridecyl ether acetic acid salt, a polyoxyethylene lauryl ether propionic acid salt, and an alkyl glycol acetic acid salt can be used.

Examples of the alkenyl succinic acid salt include an alkenyl succinic acid salt (mono salts or disalts) containing an alkenyl group having 8 to 22 carbon atoms.

Examples of the N-acyl amino acid salt include an N-acyl glutamic acid salt, an N-acyl aspartic acid salt, an N-acyl-β-alanine salt, an N-acyl methyl alanine salt, an N-acyl glycine salt, an N-acyl proline salt, an N-acyl sarcosine salt, and the like. Specifically, arginine N-lauroyl-β-alaninate, potassium N-lauroyl-β-alaninate, triethanolamine N-lauroyl-β-alaninate, sodium N-lauroyl-N-carboxymethyl-β-alaninate, sodium N-lauroyl glutamate, disodium N-stearoyl-L-glutamate, sodium N-lauroyl sarcosinate, and the like can be used.

Examples of the amide ether carboxylic acid salt include a fatty acid alkanol amide ether carboxylic acid salt, a polyoxyalkylene-modified alkylamide ether carboxylic acid salt, and the like.

Examples of the sulfonic acid-based anionic surfactant include an alkanesulfonic acid salt, an α-olefin sulfonic acid salt, an alkylbenzenesulfonic acid salt, an alkylnaphthalenesulfonic acid salt, an alkyldiphenyl ether disulfonic acid salt, an alkylglycidyl ether sulfonic acid salt, a lignin sulfonic acid salt, an α-sulfo fatty acid ester salt, an acyl isethionic acid salt, an alkyl sulfo succinic acid salt, an alkyl sulfo acetic acid salt, an N-acyl methyl taurine salt, a formalin condensate-based sulfonic acid salt, a melamine sulfonic acid salt, and the like.

Examples of the alkanesulfonic acid salt include an alkanesulfonic acid salt having 8 to 18 carbon atoms, such as a 1-octanesulfonic acid salt, a 2-octanesulfonic acid salt, a 1-decanesulfonic acid salt, a 2-decanesulfonic acid salt, a 1-dodecanesulfonic acid salt, and a 2-dodecanesulfonic acid salt.

Examples of the α-olefin sulfonic acid salt include an α-olefin sulfonic acid salt having 8 to 18 carbon atoms, such as tetradecene sulfonic acid salts.

Examples of the alkylbenzenesulfonic acid salt include a p-toluenesulfonic acid salt, a dodecylbenzenesulfonic acid salt, and the like.

Examples of the alkylnaphthalenesulfonic acid salt include a monoalkylnaphthalenesulfonic acid salt, a dialkylnaphthalenesulfonic acid salt, and the like. Specifically, a methylnaphthalenesulfonic acid salt, an ethylnaphthalenesulfonic acid salt, a propylnaphthalenesulfonic acid salt, an isopropylnaphthalenesulfonic acid salt, a butylnaphthalenesulfonic acid salt, an isobutylnaphthalenesulfonic acid salt, a dimethylnaphthalenesulfonic acid salt, a diethylnaphthalenesulfonic acid salt, a diisopropylnaphthalenesulfonic acid salt, a dibutylnaphthalenesulfonic acid salt, a diisobutylnaphthalenesulfonic acid salt, a methylnonylnaphthalenesulfonic acid salt, and the like can be used.

Examples of the alkyl diphenyl ether disulfonic acid salt include an alkyl diphenyl ether disulfonic acid salt containing an alkyl group having 1 to 20 carbon atoms. Specifically, a nonyl diphenyl ether disulfonic acid salt, a dodecyl diphenyl ether disulfonic acid salt, a stearyl diphenyl ether disulfonic acid salt, and the like can be used.

Examples of the α-sulfo fatty acid ester salt include an α-sulfo fatty acid alkyl ester salt whose fatty acid residue has 8 to 18 carbon atoms. Specifically, a methyl 2-sulfo laurate salt, a polyoxyethylene fatty acid methyl ester, and the like can be used.

Examples of the acyl isethionic acid salt include a lauroyl isethionic acid salt, a coconut oil fatty acid ethyl ester sulfonic acid salt, and the like.

Examples of the alkyl sulfo succinic acid salt include: an alkyl sulfo succinic acid salt containing an alkyl group having 8 to 18 carbon atoms, such as a dioctyl sulfo succinic acid salt, a di-2-ethylhexyl sulfo succinic acid salt, and a lauryl sulfo succinic acid salt; a polyoxyethylene alkyl sulfo succinic acid salt having 8 to 18 carbon atoms, such as a polyoxyethylene lauryl sulfo succinic acid salt; and the like.

Examples of the alkyl sulfo acetic acid salt include an alkyl sulfo acetic acid salt containing an alkyl group having 8 to 18 carbon atoms, such as lauryl sulfo acetic acid salts.

Examples of the N-acyl methyl taurine salt include a lauroyl methyl taurine salt, a myristoyl methyl taurine salt, a palmitoyl methyl to taurine salt, a stearoyl methyl taurine salt, a coconut oil fatty acid methyl taurine salt, and the like.

Examples of the formalin condensate-based sulfonic acid salt include a formalin condensate of naphthalene sulfonic acid salt, a formalin condensate of alkyl naphthalene sulfonic acid salt, a formalin condensate of melamine sulfonic acid, a formalin condensate of alkyl melamine sulfonic acid, and the like.

Examples of the sulfuric acid-based anionic surfactant include an alkyl sulfuric acid salt, an alkyl ether sulfuric acid salt, an alkyl aryl ether sulfuric acid salt, a fatty acid alkanolamide sulfuric acid salt, a fatty acid monoglyceride sulfuric acid salt, and the like.

Examples of the alkyl sulfuric acid salt include a lauryl sulfuric acid salt, a stearyl sulfuric acid salt, a cetyl sulfuric acid salt, and the like.

Examples of the alkyl ether sulfuric acid salt include a POE lauryl ether sulfuric acid salt, a POE tridecyl ether sulfuric acid salt, and the like.

Examples of the alkyl aryl ether sulfuric acid salt include polyoxyethylene nonylphenyl ether sulfuric acid salts, and the like.

Examples of the fatty acid alkanolamide sulfuric acid salt include polyoxyethylene alkyl coconut oil fatty acid monoethanolamide sulfuric acid salts, and the like.

Examples of the fatty acid monoglyceride sulfuric acid salt include hydrogenated coconut oil fatty acid glyceryl sulfuric acid salts, and the like.

Examples of the phosphoric acid-based anionic surfactant include an alkyl phosphoric acid salt, a polyoxyalkylene alkyl ether phosphoric acid salt, an alkyl aryl ether phosphoric acid salt, a fatty acid amide ether phosphoric acid salt, a glycerin fatty acid ester monophosphoric acid salt, and the like.

Examples of the alklyl phosphoric acid salt include a lauryl phosphoric acid salt, a myristyl phosphoric acid salt, a palmityl phosphoric acid salt, a stearyl phosphoric acid salt, and the like.

Examples of the polyoxyalkylene alkyl ether phosphoric acid salt include a polyoxyethylene lauryl ether phosphoric acid salt, a polyoxyethylene alkyl (C12-15) ether phosphoric acid salt, a polyoxyethylene cetyl ether phosphoric acid salt, a polyoxyethylene oleyl ether phosphoric acid salt, a polyoxyethylene stearyl ether phosphoric acid salt, and the like.

Examples of the alkyl aryl ether phosphoric acid salt include polyoxyethylene nonylphenyl ether phosphoric acid salts, and the like.

Examples of the fatty acid amide ether phosphoric acid salt include polyoxyethylene alkyl monoethanolamide phosphoric acid salts, and the like.

Examples of the nonionic surfactant include: a polyoxyalkylene alkyl ether that is an alkylene oxide adduct of an aliphatic alcohol; a polyoxyalkylene phenyl ether or a polyoxyalkylene alkylphenyl ether (polyoxyalkylene aryl ether) that is an alkylene oxide adduct of an aromatic alcohol; a glycerin fatty acid ester; a polyoxyalkylene glycerin fatty acid ester that is an alkylene oxide adduct of a glycerin fatty acid ester; a polyoxyalkylene pentaerythritol fatty acid ester that is an alkylene oxide adduct of a pentaerythritol fatty acid ester; a polyoxyalkylene fatty acid ester that is an alkylene oxide adduct of a fatty acid; a sorbitan fatty acid ester; a polyoxyalkylene sorbitan fatty acid ester that is an alkylene oxide adduct of a sorbitan fatty acid ester; a sorbitan fatty acid ester; a sucrose fatty acid ester; a pentaerythritol fatty acid ester; a polyoxyalkylene alkyl amine that is an alkylene oxide adduct of an aliphatic amine; a polyoxyalkylene fatty acid amide that is an alkylene oxide adduct of a fatty acid amide; polyoxyalkylene (hydrogenated) castor oil, a polyoxyalkylene-modified diorganopolysiloxane; a polyglyceryl-modified silicone; a glyceryl-modified silicone; a sugar-modified silicone; a perfluoropolyether-based surfactant; a polyoxyethylene-polyoxypropylene block copolymer; an alkylpolyoxyethylene-polyoxypropylene block copolymer ether; and the like. The nonionic surfactant contained in the dispersant may be only one type, or two or more types.

Examples of the polyoxyalkylene alkyl ether include a polyoxyethylene alkyl ether, a polyoxypropylene alkyl ether, a polyoxyethylene polyoxypropylene alkyl ether, and the like.

Examples of the polyoxyalkylene aryl ether include polyoxyethylene phenyl ether, polyoxyethylene distyryl phenyl ether, polyoxyethylene tristyryl phenyl ether, polyoxyethylene polyoxypropylene distyryl phenyl ether, polyoxyethylene polyoxypropylene tristyryl phenyl ether, and the like.

Examples of the (poly)glycerin fatty acid ester include a (poly)glycerin monofatty acid ester, a (poly)glycerin difatty acid ester, a (poly)glycerin trifatty acid ester, and the like. Examples of the (poly)glycerin monofatty acid ester include glycerin monocaprylate, glycerin monostearate, glycerin monobehenate, and the like. Examples of the (poly)glycerin difatty acid ester include glycerin distearate, glycerin dibehenate, and the like. Examples of the (poly)glycerin trifatty acid ester include glycerin tristearate, glycerin tribehenate, and the like.

Examples of the polyoxyalkylene glycerin fatty acid ester include polyoxyethylene glycerin monostearate, polyoxyethylene glycerin monooleate, and the like.

Examples of the polyoxyalkylene fatty acid ester include a polyoxyethylene fatty acid ester, a polyoxypropylene fatty acid ester, and the like. Examples of the polyoxyethylene fatty acid ester include polyoxyethylene monostearic acid ester, polyoxyethylene distearic acid ester, polyoxyethylene monooleic acid ester, polyoxyethylene dioleic acid ester, and the like.

Examples of the sorbitan fatty acid ester include a sorbitan monofatty acid ester, a sorbitan sesquifatty acid ester, a sorbitan difatty acid ester, a sorbitan trifatty acid ester, a sorbitan tetrafatty acid ester, and the like. Examples of the sorbitan monofatty acid ester include sorbitan monopalmitate, sorbitan monostearate, sorbitan monoisostearate, sorbitan monooleate, sorbitan monobehenate, and the like. Examples of the sorbitan difatty acid ester include sorbitan dipalmitate, sorbitan distearate, sorbitan dibehenate, and the like. Examples of the sorbitan trifatty acid ester include sorbitan tripalmitate, sorbitan tristearate, sorbitan tribehenate, and the like.

Examples of the polyoxyalkylene sorbitan fatty acid ester include polyoxyethylene sorbitan monostearate, polyoxyethylene sorbitan monooleate, and the like.

Examples of the polyoxyalkylene alkyl amine include a polyoxyethylene alkyl amine, a polyoxypropylene alkyl amine, and the like.

Examples of the polyoxyalkylene fatty acid amide include polyoxyethylene lauric acid monoethanolamide, polyoxyethylene lauric acid monoethanolamide, polyoxyethylene lauric acid monoethanolamide, polyoxyethylene lauric acid monoethanolamide, polyoxyethylene coconut oil fatty acid monoethanolamide, polyoxyethylene coconut oil fatty acid monoethanolamide, polyoxyethylene coconut oil fatty acid monoethanolamide, polyoxyethylene coconut oil fatty acid monoethanolamide, polyoxypropylene myrystic acid monoethanolamide, and the like.

From a viewpoint of the stability of the deodorant-containing processing liquid, a content of the dispersant contained in the deodorant-containing processing liquid according to the embodiment of the present invention is preferably in a range from 0.1 to 60 parts by mass, more preferably from 0.3 to 50 parts by mass, and further preferably from 0.5 to 40 parts by mass based on 100 parts by mass of the inorganic carrier.

The deodorant-containing processing liquid according to the embodiment of the present invention is a dispersion liquid in which a part or all of the above-described components are dispersed in the dispersion medium. A content of the dispersion medium is not particularly limited and is adjusted so that a content of the acid salt of aminoguanidine is preferably in a range from 0.15 to 30 parts by mass, and more preferably from 1 to 20 part by mass based on an amount of the deodorant-containing processing liquid.

As described above, the deodorant-containing processing liquid according to the embodiment of the present invention may contain other components such as other deodorant that chemically adsorbs aldehyde-based gases or other odorous gases (e.g., sulfur-based gases such as hydrogen sulfide and methyl mercaptan; and organic acid gases such as acetic acid, isovaleric acid, and butyric acid) and an additive. Hereinafter, the other components will be described.

Examples of the deodorant that adsorbs aldehyde-based gases include a hydrazide, an azole having an amino group, and the like.

A structure of the hydrazide is not particularly limited. The hydrazide may be any one of a monohydrazide having one hydrazide group in molecule, a dihydrazide having two hydrazide groups in molecule, and a polyhydrazide having three or more hydrazide groups in molecule.

Examples of the monohydrazide include a compound represented by the following general formula (3):

R¹—CO—NHNH₂ (3)

(In the formula, R¹ is a substituted or unsubstituted hydrocarbon group having 3 or more carbon atoms).

Examples of the monohydrazide include propanoic acid hydrazide, butanoic acid hydrazide, pentanoic acid hydrazide, hexanoic acid hydrazide, heptanoic acid hydrazide, octanoic acid hydrazide, nonanoic acid hydrazide, decanoic acid hydrazide, dodecanoic acid hydrazide, pentadecanoic acid hydrazide, 4-methyl benzoic acid hydrazide, phthalic acid monohydrazide, isophthalic acid monohydrazide, terephthalic acid monohydrazide, naphthoic acid hydrazide, salicylic acid hydrazide, p-hydroxybenzoic acid hydrazide, 3-hydroxy-2-naphthoic acid hydrazide, 6-hydroxy-2-naphthoic acid hydrazide, and the like.

Examples of the dihydrazide include a compound represented by the following general formula (4):

H₂NHN—X—NHNH₂ (4)

(In the formula, X is a —CO— group or a —CO—R²—CO— group and R² is a substituted or unsubstituted alkylene group and cycloalkylene group having 3 or more carbon atoms, or arylene group).

Examples of the alkylene group represented by R² in the above general formula (4) include a trimethylene group, a butylene group, a pentylene group, a hexylene group, a heptylene group, an octylene group, a nonylene group, a decamethylene group, an undecamethylene group, and the like. Examples of the cycloalkylene group include a cyclobutylene group, a cyclopentylene group, a cyclohexylene group, a cycloheptylene group, a cyclooctylene group, and the like. Examples of the arylene group include a phenylene group, a biphenylene group, a naphthylene group, an anthrylene group, a pentacenylene group, a perylenylene group, a picenylene group, a pyrenylene group, a fluorenylene group, a chrysenylene group, a phenanthrylene group, and the like.

Examples of the dihydrazide include succinic acid dihydrazide, glutaric acid dihydrazide, adipic acid dihydrazide, azelaic acid dihydrazide, sebacic acid dihydrazide, 1,12-dodecanedicarbohydrazide, 1,18-octadecanedicarbohydrazide, maleic acid dihydrazide, fumaric acid dihydrazide, diglycol acid dihydrazide, tartaric acid dihydrazide, malic acid dihydrazide, dimer acid dihydrazide, phthalic acid dihydrazide, isophthalic acid dihydrazide, terephthalic acid dihydrazide, 2-methyl terephthalic acid dihydrazide, 5-tert-butylisophthalic acid dihydrazide, 1,4-naphthalenedicarbohydrazide, 2,6-naphthalenedicarbohydrazide (2,6-naphthoic acid dihydrazide), 4,4′-bisbenzenedihydrazide, 5-hydroxyisophthalic acid dihydrazide, 2-ethoxyterephthalic acid dihydrazide, 3-methoxyphthalic acid dihydrazide, 5-butoxyisophthalic acid dihydrazide, 2-phenoxyterephthalic acid dihydrazide, 4,6-dimethoxyisphthalic acid dihydrazide, 2,3-bis(benzyloxy)terephthalic acid dihydrazide, 4,4′-oxybis(phthalic acid dihydrazide), hydroquinone diglycol acid dihydrazide, resorcinol diglycol acid dihydrazide, catechol diglycol acid dihydrazide, 4,4′-ethylidene bisphenol-diglycol acid dihydrazide, 4,4′-vinylidene bisphenol-diglycol acid dihydrazide, 3-aminophthalic acid dihydrazide, 3-nitrophthalic acid dihydrazide, 5-(dibenzylamino)isophtahlic acid dihydrazide, and the like.

Examples of the polyhydrazide include a compound represented by the following general formula (5):

R³—(CO—NHNH₂)ₙ (5)

(In the formula, R³ is a substituted or unsubstituted n-valent hydrocarbon group, and n is an integer of 3 or more).

Examples of the polyhydrazide include a trihydrazide such as 1,2,4-benzenetricarbohydrazide and pyromellitic acid trihydrazide; a tetrahydrazide such as pyromellitic acid tetrahydrazide, 1,4,5,8-naphthalenetetracarbohydrazide and 5,5′-ethylenebisoxybis(isophthalic acid dihydrazide); and the like.

Examples of the azole having an amino group include 3-aminopyrazole, 5-amino-3-methylpyrazole, 3-amino-1,2,4-triazole, 4-amino-1,2,4-triazole, 3,5-diamino-1,2,4-triazole, 5-amino-3-mercapto-1,2,4-triazole, 3-amino-5-phenyl-1,2,4-triazole, and the like.

Examples of the deodorant that adsorbs sulfur-based gases include metallic copper, a compound containing a copper element (e.g., copper oxide, copper silicate), metallic zinc, a compound containing a zinc element (e.g., zinc oxide, zinc silicate), a compound containing a manganese element, and the like.

Examples of the deodorant that adsorbs organic acid gases include hydrous zirconium oxide, and the like.

An average particle diameter of each of the above-described other deodorants is preferably in a range from 0.01 to 50 μm, and more preferably from 0.02 to 20 μm from a viewpoint of handleability, adsorption performance, etc.

When the deodorant-containing processing liquid according to the embodiment of the present invention contains other deodorant, an upper limit of a content of the other deodorant is preferably 5,000 parts by mass, and more preferably 3,000 parts by mass based on 100 parts by mass of the acid salt of aminoguanidine.

Examples of the additive include a water-soluble or hydrophilic organic solvent, a viscosity adjuster, an antifoaming agent, a colorant, an air freshener, an antimicrobial agent, an antivirus agent, an anti-allergen agent, an antiseptic agent, and the like.

Examples of the organic solvent include methanol, ethanol, dimethyl formamide, dimethyl acetamide, dimethyl sulfoxide, tetrahydrofuran, acetone, and the like.

The pH of the deodorant-containing processing liquid according to the embodiment of the present invention is preferably in a range from 1 to 7, and more preferably from 1.5 to 6, from a viewpoint of stability.

The deodorant-containing processing liquid according to the embodiment of the present invention has excellent stability in spite of containing a deodorant at high concentration. A viscosity (25° C., 6 rpm) of the deodorant-containing processing liquid according to the embodiment of the present invention as measured with a B-type viscometer is preferably in a range from 50 to 1,000 cps, and more preferably from 100 to 800 cps. When having a viscosity within the above range, the deodorant-containing processing liquid according to the embodiment of the present invention is easy to handle and has excellent applicability onto a base. Therefore, it is possible to efficiently produce a deodorizing product for aldehyde-based gases whose impregnation amount of a deodorant is high.

A preparation method of the deodorant-containing processing liquid according to the embodiment of the present invention is not particularly limited. Preferable preparation methods are as follows:

(1) a method in which the components are mixed at a time or in several batches;

(2) a method in which a complex obtained by allowing the inorganic carrier to cany the acid salt of aminoguanidine, a dispersant containing liquid (aqueous solution or dispersion liquid) containing the dispersant including the above-described surfactant and water, the adhesive resin, and if necessary, water are mixed at a time or in several batches;

(3) a method in which a complex obtained by allowing the inorganic carrier to cany the acid salt of aminoguanidine, the dispersant including the above-described surfactant, a resin dispersion liquid or aqueous solution containing the adhesive resin and water, and if necessary, water are mixed at a time or in several batches;

(4) a method in which a complex obtained by allowing the inorganic carrier to carry the acid salt of aminoguanidine, a dispersant containing liquid (aqueous solution or dispersion liquid) containing the dispersant including the above-described surfactant and water, a resin dispersion liquid or aqueous solution containing the adhesive resin and water, and if necessary, water are mixed at a time or in several batches; and

(5) a method in which a complex obtained by allowing the inorganic carrier to carry the acid salt of aminoguanidine, a resin emulsion (resin dispersion liquid) produced in the presence of the above-described surfactant, and if necessary, the above-described surfactant and/or water are mixed at a time or in several batches.

Among these, methods (2) and (4) are preferable.

As described above, the deodorant-containing processing liquid according to the embodiment of the present invention is suitable as a raw material for producing a deodorizing product by impregnating a base with a deodorant. A method for producing the deodorizing product is not particularly limited and is usually selected depending on the shape etc. of the base.

The production method of a deodorizing product according to the embodiment of the present invention includes a step for applying the above-described deodorant-containing processing liquid according to the embodiment of the present invention onto a base to form a coating film on a surface of the base (hereinafter, referred to as “coating step”) and a step for drying the coating film (hereinafter, referred to as “drying step”).

In the coating step, a coating film is formed on the surface of the base by, for example, padding, dipping, coating, spraying, or printing depending on the shape etc. of the base. The coating film may be a film obtained by infiltrating the deodorant-containing processing liquid into the base so that a continuous phase is formed in the surface layer and inside of the base.

The base is not particularly limited. The base may be an article containing an inorganic material, an organic material, or a material obtained by combining them. A shape of the base is not particularly limited. Examples of the base that can be used include: resin molded articles (including foamed resin molded articles) such as films, particles, and general molded articles; fibers; and fiber products containing fibers, such as nonwoven fabrics and woven fabrics.

In the drying step, a medium including water is removed from the coating film by closed heating, warm air heating, or the like depending on the shape etc. of the base to form a deodorant-containing part (deodorant coating).

A drying temperature is preferably in a range from 60° C. to 150° C., and more preferably from 80° C. to 130° C., and a drying time is preferably in a range from 2 minutes to 12 hours, and more preferably from 5 minutes to 2 hours.

As described above, in the embodiment of the present invention the deodorant-containing processing liquid is used which is stable and has a high content of the acid salt of aminoguanidine with respect to the content of the inorganic carrier. Therefore, it is not necessary to repeatedly perform the coating step and the drying step to produce a deodorizing product having a desired deodorant content, that is, it is possible to efficiently produce a desired deodorizing product.

A deodorizing product obtained by the present invention will be exemplified later.

A deodorizing filter medium according to the embodiment of the present invention is an article including a sheet-shaped base containing fiber and a deodorant-containing part joined to a surface of the fiber, and having an air permeable from one surface side to other surface side. The deodorant-containing part contains an acid salt of aminoguanidine, an inorganic carrier carrying the acid salt of aminoguanidine, an adhesive resin, and a surfactant, a content of the acid salt of aminoguanidine is 15 parts or more by mass based on 100 parts by mass of the content of the inorganic carrier, and the surfactant is at least one selected from an anionic surfactant and a nonionic surfactant.

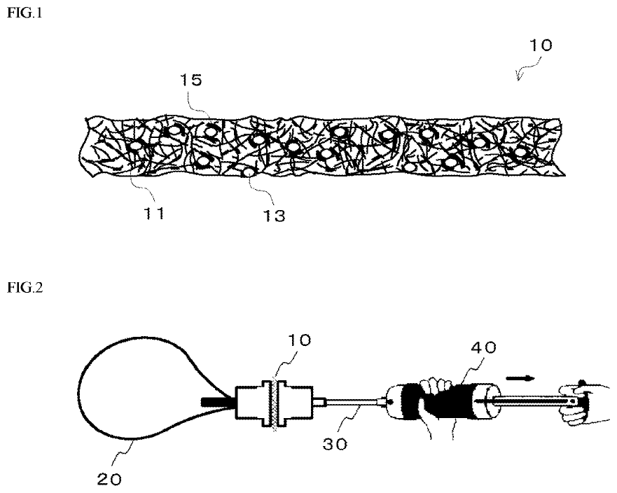

FIG. 1 shows, as a preferred mode of the deodorizing filter medium according to the embodiment of the present invention, a deodorizing filter medium 10 in which fiber 11 and a complex 13 obtained by allowing an inorganic carrier to carry an acid salt of aminoguanidine are joined via an adhesive part 15 containing an adhesive resin and a surfactant. In FIG. 1, the complex 13 and the adhesive part 15 form the deodorant-containing part according to the embodiment of the present invention. A structure of the deodorizing filter medium according to the embodiment of the present invention is not limited to one shown in FIG. 1. For example, the deodorizing filter medium may have a structure (not shown) in which the complex 13 is localized in the center to form a deodorant layer or a structure (not shown) in which the complex 13 is localized on its one surface side to form a deodorant layer.

The base is a sheet-shaped article that contains fiber and has an air permeable from one surface side to other surface side, and may be formed of either a woven fabric or a nonwoven fabric. However, the base is preferably formed of a nonwoven fabric because a desired thickness can easily be set and the ability to allow air to pass therethrough can easily be controlled.

The fiber is preferably mainly made of a resin, and examples of the resin include a polyester, a polyethylene, a polypropylene, a polyvinyl chloride, a polyacrylic acid, a polyamide, a polyvinyl alcohol, a polyurethane, a polyvinyl ester, a polymethacrylate, a rayon, and the like. Among these, a polyester, a polyethylene, a polypropylene, a polyamide, a polyvinyl alcohol, and a polyurethane are preferable. The nonwoven fabric may be one formed of fibers containing only one kind of resin or one formed of two or more kinds of resin fibers. The nonwoven fabric may be one in which fibers are interlaced.

The deodorant-containing part according to the embodiment of the present invention contains a complex obtained by allowing an inorganic carrier to carry an acid salt of aminoguanidine, an adhesive resin, and a surfactant. Preferred materials for the acid salt of aminoguanidine, the inorganic carrier, the adhesive resin, and the surfactant are as described above with reference to the deodorant-containing processing liquid according to the embodiment of the present invention.

A content of the acid salt of aminoguanidine at the deodorant-containing part is 15 parts by mass or more, preferably in a range from 15 to 200 parts by mass, more preferably from 20 to 150 parts by mass, and further preferably from 25 to 100 parts by mass, based on 100 parts by mass of the content of the inorganic carrier from a viewpoint of deodorizing odorous gases including aldehyde-based gases.

A content ratio of the acid salt of aminoguanidine is preferably in a range from 1% to 50% by mass, more preferably from 3% to 40%, and further preferably from 5% to 30% by mass, based on an amount of the base from a viewpoint of deodorizing odorous gases including aldehyde-based gases.

A content of the adhesive resin at the deodorant-containing part is preferably in a range from 10 to 300 parts by mass, more preferably from 15 to 200 parts by mass, and further preferably from 20 to 150 parts by mass, based on 100 parts by mass of a total amount of the acid salt of aminoguanidine and the inorganic carrier from a viewpoint of adhesiveness between the complex obtained by allowing the inorganic carrier to carry the acid salt of aminoguanidine and the fiber.

A content of the surfactant at the deodorant-containing part is preferably in a range from 0.1 to 60 parts by mass, more preferably from 0.3 to 50 parts by mass, and further preferably from 0.5 to 40 parts by mass, based on 100 parts by mass of the inorganic carrier.

A weight per unit area of the deodorizing filter medium according to this embodiment of the present invention is preferably in a range from 25 to 200 g/m², and more preferably from 40 to 150 g/m², from viewpoints of a sufficient deodorizing effect on aldehyde-based gases and ability to allow air to pass therethrough.

The air permeability of the deodorizing filter medium according to the embodiment of the present invention is preferably in a range from 10 to 500 cm³/(cm²·s), and more preferably from 50 to 300 cm³/(cm²·s), from a viewpoint of a sufficient deodorizing effect on aldehyde-based gases. It is to be noted that the air permeability is determined by a method based on JIS L1096.

A production method of the deodorizing filter medium according to the embodiment of the present invention is not particularly limited. Applicable methods are as follows:

(1) a method in which the above-described deodorant-containing processing liquid according to the embodiment of the present invention is applied (by, for example, dipping, spraying, or padding) onto a base formed of a woven or unwoven fabric containing fibers and then dried so that a complex obtained by allowing the inorganic carrier to carry the acid salt of aminoguanidine is adhered to the surface of the fibers contained in the woven or nonwoven fabric; and

(2) a method in which a liquid containing an adhesive resin, a surfactant, and a medium is applied (by, for example, dipping, spraying, or padding) onto a base formed of a woven or nonwoven fabric containing fibers, a complex obtained by allowing an inorganic carrier to carry an acid salt of aminoguanidine is then sprayed onto the base, and the medium is remover so that the complex obtained by allowing an inorganic carrier to carry an acid salt of aminoguanidine is adhered to the surface of the fibers contained in the woven or nonwoven fabric.

A deodorizing filter unit according to the embodiment of the present invention includes the above-described deodorizing filter medium according to the embodiment of the present invention and a carrying member that carries the deodorizing filter medium. That is, the deodorizing filter unit according to the embodiment of the present invention is a composite product including the deodorizing filter medium and the carrying member.

A shape and constituent material of the carrying member are not particularly limited and can appropriately be selected depending on the intended use.

A shape of the deodorizing filter medium is also not particularly limited and can also be appropriately selected depending on the intended use. For example, the deodorizing filter medium may be pleated to have a zigzag shape, or the two or more deodorizing filter media may three-dimensionally be layered to have a multi-layer structure.

Examples of the structure of the deodorizing filter unit according to the embodiment of the present invention are as follows:

(1) a structure in which the deodorizing filter medium is fitted in the carrying member having a frame shape;

(2) a structure in which the deodorizing filter medium is joined to the carrying member; and

(3) a structure in which the carrying member having a ring shape is provided between fibers at the periphery of the deodorizing filter medium.

Depending on the purpose or intended use, the deodorizing filter unit according to the embodiment of the present invention may further include another member such as a dust collection filter medium, a disinfectant filter medium, an anti-allergen filter medium, and an anti-virus filter medium.

A deodorizing apparatus according to the embodiment of the present invention includes the above-described deodorizing filter medium according to the embodiment of the present invention, and may include the above-described deodorizing filter unit according to the embodiment of the present invention.

The deodorizing apparatus according to the embodiment of the present invention preferably includes a main body, an odorous gas inlet provided in the main body to introduce odorous gas from the outside of the main body, a deodorizing filter medium provided inside the main body, a gas outlet provided in the main body to discharge gas cleaned by the deodorizing filter medium, and an exhaust fan provided inside the main body to forcibly discharge the cleaned gas through the gas outlet.

### EXAMPLES

Hereinafter, the present invention will be described in more detail with reference to Examples, but is not limited to these Examples. In the following, it is noted that “%” refers to % by mass unless otherwise mentioned.

### 1. Raw Materials of Deodorant-Containing Processing Liquid

Raw materials used for producing deodorant-containing processing liquids are as follows.

### 1-1. Deodorant Composition

A complex deodorant including a complex was obtained by the above-described method using an aqueous solution of a hydrochloric acid salt or sulfuric acid salt of aminoguanidine and aluminum silicate (molar ratio SiO₂:Al₂O₃=9:1) having a BET specific surface area of 600 m²/g and an average particle diameter of 10 μm or silica gel having a BET specific surface area of 700 m²/g and an average particle diameter of 5 μm as an inorganic carrier, and was then, if necessary, mixed with hydrous zirconium oxide or zinc oxide as other deodorant to prepare deodorant compositions (A1) to (A11) shown in Table 1. After that, these deodorant compositions (A1) to (A11) were used as raw materials for producing deodorant-containing processing liquids.

TABLE 1

| | | Deodorant composition | | | | | | | | | | |
|---|---|---|---|---|---|---|---|---|---|---|---|---|
| | | A1 | A2 | A3 | A4 | A5 | A6 | A7 | AS | A9 | A10 | A11 |
| Hydrochloric acid salt of aminoguanidine | (parts by mass) | 6 | 5 | 3 | 4 | 5 | 4 | 3 | | | | |
| Sulfuric acid salt of aminoguanidine | (parts by mass) | | | | | | | | 6 | 4 | 3 | 4 |
| Aluminum silicate | (parts by mass) | 6 | 10 | 12 | 12 | | | | 6 | 12 | 12 | |
| Silica | (parts by mass) | | | | | 10 | 10 | 12 | | | | 12 |
| Hydrous zirconium oxide | (parts by mass) | | | | 2 | | | | | | | |
| Zinc oxide | (parts by mass) | | | | | | | | | | 2 | |

| | | | | | | | | | | | |
|---|---|---|---|---|---|---|---|---|---|---|---|
| Content ratio (parts by mass) of acid salt of aminoguanidine | 100 | 50 | 25 | 33 | 50 | 40 | 25 | 100 | 33 | 25 | 33 |
| based on 100 parts by mass of inorganic carrier | | | | | | | | | | | |

### 1-2. Adhesives

Emulsions containing an adhesive resin were used.

(1) Emulsion (B1)

An ethylene-vinyl acetate copolymer emulsion was used. The solid content concentration of the adhesive resin is 57%.

### (2) Emulsion (B2)

An ethylene-vinyl chloride copolymer emulsion was used. The solid content concentration of the adhesive resin is 50%.

### (3) Emulsion (B3)

An acrylic resin emulsion was used. The solid content concentration of the adhesive resin is 40%.

### (4) Emulsion (B4)

A urethane resin emulsion was used. The solid content concentration of the adhesive resin is 30%.

### (5) Emulsion (B5)

A chlorinated polyolefin emulsion was used. The solid content concentration of the adhesive resin is 30%.

### 1-3. Dispersants

Dispersant containing liquids containing a surfactant were used.

### (1) Dispersant Containing Liquid (C1)

A water-soluble dispersant containing an anionic surfactant (acid value: 1.5 mgKOH/g) was used. The pure content of the surfactant is 40%.

### (2) Dispersant Containing Liquid (C2)

A water-soluble dispersant containing an anionic surfactant (acid value: 10 mgKOH/g) was used. The pure content of the surfactant is 40%.

### (3) Dispersant Containing Liquid (C3)

A water-soluble dispersant containing a nonionic surfactant was used. The pure content of the surfactant is 40%.

### (4) Dispersant Containing Liquid (C4)

A water-soluble dispersant containing a nonionic surfactant was used. The pure content of the surfactant is 60%.

### (5) Dispersant Containing Liquid (C5)

A water-soluble dispersant mainly containing a cationic surfactant (quaternary ammonium salt) was used. The pure content of the surfactant is 63%.

### (6) Dispersant Containing Liquid (C6)

A water-soluble dispersant containing an amphoteric surfactant (acid value: 30 mgKOH/g, amine value: 20 mgKOH/g) was used. The pure content of the surfactant is 98%.

### (7) Dispersant Containing Liquid (C7)

A water-soluble dispersant containing an amphoteric surfactant (acid value: 94 mgKOH/g, amine value: 94 mgKOH/g) was used. The pure content of the surfactant is 81%.

### (8) Dispersant Containing Liquid (C8)

A water-soluble dispersant containing an amphoteric surfactant (acid value: 8 mgKOH/g, amine value: 18 mgKOH/g) was used. The pure content of the surfactant is more than 98.5%.

### 2. Production and Evaluation of Deodorant-Containing Processing Liquids

The deodorant compositions shown in Table 1, emulsions, dispersant containing liquids, and water were used to produce deodorant-containing processing liquids, and various physical properties were evaluated.

### Example 1-1

15 parts of the deodorant composition (A3), 9.1 parts of the emulsion (B1), 1.5 parts of the dispersant containing liquid (C1), and 74.4 parts of pure water were mixed to produce a deodorant-containing processing liquid (L1) (see Table 2). Then, the pH of the deodorant-containing processing liquid at 25° C. was measured, and the viscosity of the deodorant-containing processing liquid at 25° C. was measured using a B-type viscometer (manufactured by Toki Sangyo Co., Ltd, Type BL, No. 3 rotor). These results are also shown in Table 2.

### Example 1-2

15 parts of the deodorant composition (A2), 10 parts of the emulsion (B2), 2.5 parts of the dispersant containing liquid (C4), and 72.5 parts of pure water were mixed to produce a deodorant-containing processing liquid (L2) (see Table 2). Then, the pH and viscosity of the deodorant-containing processing liquid were measured in the same manner as those in Example 1-1 (see Table 2).

### Example 1-3

12 parts of the deodorant composition (A1), 15 parts of the emulsion (B3), 2 parts of the dispersant containing liquid (C3), and 71 parts of pure water were mixed to produce a deodorant-containing processing liquid (L3) (see Table 2). Then, the pH and viscosity of the deodorant-containing processing liquid were measured in the same manner as those in Example 1-1 (see Table 2).

### Example 1-4

18 parts of the deodorant composition (A4), 8.2 parts of the emulsion (B1), 1.5 parts of the dispersant containing liquid (C1), and 72.3 parts of pure water were mixed to produce a deodorant-containing processing liquid (L4) (see Table 2). Then, the pH and viscosity of the deodorant-containing processing liquid were measured in the same manner as those in Example 1-1 (see Table 2).

### Example 1-5

15 parts of the deodorant composition (A5), 16.7 parts of the emulsion (B4), 2 parts of the dispersant containing liquid (C1), and 66.3 parts of pure water were mixed to produce a deodorant-containing processing liquid (L5) (see Table 2). Then, the pH and viscosity of the deodorant-containing processing liquid were measured in the same manner as those in Example 1-1 (see Table 2).

### Example 1-6

17 parts of the deodorant composition (A10), 21.3 parts of the emulsion (B3), 2 parts of the dispersant containing liquid (C3), and 59.7 parts of pure water were mixed to produce a deodorant-containing processing liquid (L6) (see Table 2). Then, the pH and viscosity of the deodorant-containing processing liquid were measured in the same manner as those in Example 1-1 (see Table 2).

### Example 1-7

16 parts of the deodorant composition (A9), 10 parts of the emulsion (B3), 1.5 parts of the dispersant containing liquid (C2), and 72.5 parts of pure water were mixed to produce a deodorant-containing processing liquid (L7) (see Table 2). Then, the pH and viscosity of the deodorant-containing processing liquid were measured in the same manner as those in Example 1-1 (see Table 2).

### Example 1-8

16 parts of the deodorant composition (A11), 13.3 parts of the emulsion (B5), 1.5 parts of the dispersant containing liquid (C2), and 69.2 parts of pure water were mixed to produce a deodorant-containing processing liquid (L8) (see Table 2). Then, the pH and viscosity of the deodorant-containing processing liquid were measured in the same manner as those in Example 1-1 (see Table 2).

### Comparative Example 1-1

15 parts of the deodorant composition (A3), 9.1 parts of the emulsion (B1), and 75.9 parts of pure water were mixed to produce a deodorant-containing processing liquid (L9) (see Table 2). Then, the pH and viscosity of the deodorant-containing processing liquid were measured in the same manner as those in Example 1-1 (see Table 2).

### Comparative Example 1-2

15 parts of the deodorant composition (A7), 18.8 parts of the emulsion (B3), 1 part of the dispersant containing liquid (C5), and 65.2 parts of pure water were mixed to produce a deodorant-containing processing liquid (L10) (see Table 2). Then, the pH and viscosity of the deodorant-containing processing liquid were measured in the same manner as those in Example 1-1 (see Table 2).

### Comparative Example 1-3

14 parts of the deodorant composition (A6), 6.4 parts of the emulsion (B1), 1 part of the dispersant containing liquid (C7), and 78.6 parts of pure water were mixed to produce a deodorant-containing processing liquid (L11) (see Table 2). Then, the pH and viscosity of the deodorant-containing processing liquid were measured in the same manner as those in Example 1-1 (see Table 2).

### Comparative Example 1-4

15 parts of the deodorant composition (A5), 16.7 parts of the emulsion (B4), 2 parts of the dispersant containing liquid (C8), and 66.3 parts of pure water were mixed to produce a deodorant-containing processing liquid (L12) (see Table 2). Then, the pH of the deodorant-containing processing liquid was measured in the same manner as those in Example 1-1 (see Table 2). The viscosity could not be measured because the raw materials were agglomerated.

### Comparative Example 1-5

16 parts of the deodorant composition (A9), 10 parts of the emulsion (B3), 1.5 parts of the dispersant containing liquid (C6), and 72.5 parts of pure water were mixed to produce a deodorant-containing processing liquid (L13) (see Table 2). Then, the pH and viscosity of the deodorant-containing processing liquid were measured in the same manner as those in Example 1-1 (sec Table 2).

### Comparative Example 1-6

12 parts of the deodorant composition (A8), 10 parts of the emulsion (B5), 1 part of the dispersant containing liquid (C5), and 77 parts of pure water were mixed to produce a deodorant-containing processing liquid (L14) (see Table 2). Then, the pH and viscosity of the deodorant-containing processing liquid were measured in the same manner as those in Example 1-1 (see Table 2).

TABLE 2

| | | Examples | | | | | | | | Comparative Examples | | | | | |
|---|---|---|---|---|---|---|---|---|---|---|---|---|---|---|---|
| | | 1-1 | 1-2 | 1-3 | 1-4 | 1-5 | 1-6 | 1-7 | 1-8 | 1-1 | 1-2 | 1-3 | 1-4 | 1-5 | 1-6 |
| Processing | | L1 | L2 | L3 | L4 | L5 | L6 | L7 | L8 | L9 | L10 | L11 | L12 | L13 | L14 |
| liquids | | | | | | | | | | | | | | | |
| Deodorant | A1 | | | 12 | | | | | | | | | | | |
| composition | A2 | | 15 | | | | | | | | | | | | |
| (parts) | A3 | 15 | | | | | | | | 15 | | | | | |
| | A4 | | | | 18 | | | | | | | | | | |
| | A5 | | | | | 15 | | | | | | | 15 | | |
| | A6 | | | | | | | | | | | 14 | | | |
| | A7 | | | | | | | | | | 15 | | | | |
| | A8 | | | | | | | | | | | | | | 12 |
| | A9 | | | | | | | 16 | | | | | | 16 | |
| | A10 | | | | | | 17 | | | | | | | | |
| | A11 | | | | | | | | 16 | | | | | | |
| Emulsion | B1 | 9.1 | | | 8.2 | | | | | 9.1 | | 6.4 | | | |
| (parts) | | (5.2) | | | (4.7) | | | | | (5.2) | | (3.6) | | | |
| | B2 | | 10 | | | | | | | | | | | | |
| | | | (5.0) | | | | | | | | | | | | |
| | B3 | | | 15 | | | 21.3 | 10 | | | 18.8 | | | 10 | |
| | | | | (6.0) | | | (8.5) | (4.0) | | | (7.5) | | | (4.0) | |
| | B4 | | | | | 16.7 | | | | | | | 16.7 | | |
| | | | | | | (5.0) | | | | | | | (5.0) | | |
| | B5 | | | | | | | | 13.3 | | | | | | 10 |
| | | | | | | | | | (4.0) | | | | | | (3.0) |
| Dispersant- | C1 | 1.5 | | | 1.5 | 2 | | | | | | | | | |
| containing | | (0.6) | | | (0.6) | (0.8) | | | | | | | | | |
| liquid | C2 | | | | | | | 1.5 | 1.5 | | | | | | |
| (parts) | | | | | | | | (0.6) | (0.6) | | | | | | |
| | C3 | | | 2 | | | 2 | | | | | | | | |
| | | | | (0.8) | | | (0.8) | | | | | | | | |
| | C4 | | 2.5 | | | | | | | | | | | | |
| | | | (1.5) | | | | | | | | | | | | |
| | C5 | | | | | | | | | | 1 | | | | 1 |
| | | | | | | | | | | | (0.63) | | | | (0.63) |
| | C6 | | | | | | | | | | | | | 1.5 | |
| | | | | | | | | | | | | | | (1.47) | |
| | C7 | | | | | | | | | | | 1 | | | |
| | | | | | | | | | | | | (0.81) | | | |
| | C8 | | | | | | | | | | | | 2 | | |
| | | | | | | | | | | | | | (>1.97) | | |

| | | | | | | | | | | | | | | |
|---|---|---|---|---|---|---|---|---|---|---|---|---|---|---|
| Water | 74.4 | 72.5 | 71 | 72.3 | 66.3 | 59.7 | 72.5 | 69.2 | 75.9 | 65.2 | 78.6 | 66.3 | 72.5 | 77 |
| (parts) | | | | | | | | | | | | | | |
| Total | 100 | 100 | 100 | 100 | 100 | 100 | 100 | 100 | 100 | 100 | 100 | 100 | 100 | 100 |
| (parts) | | | | | | | | | | | | | | |
| pH | 4.3 | 4.0 | 3.8 | 4.2 | 4.0 | 4.1 | 4.1 | 4.3 | 4.3 | 4.4 | 4.2 | 3.9 | 4.1 | 4.1 |
| Viscosity | | | | | | | | | | | | | | |
| 6 rpm | 500 | 600 | 400 | 600 | 400 | 600 | 400 | 300 | 1800 | 4000 | 2200 | Aggregated | 1600 | 3200 |
| 60 rpm | 100 | 150 | 80 | 150 | 80 | 200 | 100 | 60 | 200 | 600 | 360 | Aggregated | 250 | 400 |

(Note)
The values in brackets are solid contents (parts).

As can be seen from Table 2, Comparative Example 1-1 is an example that used no anionic or nonionic surfactant, and therefore the obtained deodorant-containing processing liquid had too high a viscosity. Comparative Examples 1-2 to 1-6 are examples that used, as a dispersant, a cationic or amphoteric surfactant, and therefore the obtained deodorant-containing processing liquid had too high a viscosity or aggregation of the raw material component occurred. On the other hand, such defect phenomena did not occur in Examples 1-1 to 1-8, and the obtained deodorant-containing processing liquids had excellent stability.

### 3. Production and Evaluation of Deodorizing Products

Various deodorizing products were produced using the deodorant-containing processing liquids shown in Table 2 and the following base, and deodorizing performances of the deodorizing products were evaluated.

### (1) Nonwoven Fabric Sheet

A chemical-bond nonwoven fabric “TN6OBT” manufactured by Kurashiki Textile Manufacturing Co., Ltd. was used. The air permeability of the nonwoven fabric measured in accordance with JIS L1096 is 310 cm³/(cm²·sec), and the weight per unit area (basis weight) of the nonwoven fabric is 60 g/m².

### (2) Paper

“TANOSEE α Eco Paper Type TR” (trade name) manufactured by OTSUKA CORPORATION was used. The basis weight of the paper is 66 g/m².

### (3) Resin Film

An easy adhesive PET film “A4300” (trade name) manufactured by TOYOBO CO., LTD. was used. The thickness of the film is 50 μm.

### Example 2-1

### (1) Production and Evaluation of Deodorizing Filter Medium

The above-described nonwoven fabric sheet was dipped in the deodorant-containing processing liquid (L1) at 25° C. (5 seconds). Then, the nonwoven fabric sheet was squeezed with a mangle and then dried by heating (130° C., 3 minutes) to obtain a deodorizing filter medium.

The obtained filter medium 10 was subjected to an aeration deodorant test (see FIG. 2) to evaluate deodorizing performance by the following method. The deodorizing filter medium 10 was set between a gas bag 20 containing air containing 200 vol ppm of formaldehyde (hereinafter, referred to as a “formaldehyde-containing gas) and a gas detector tube 30. Then, the formaldehyde-containing gas was sucked by a gas collector 40 so as to be passed through the deodorizing filter medium 10 by a suction force, and the concentration of formaldehyde in the gas passed through the deodorizing filter medium 10 was measured by the gas detector tube 30. It is to be noted that the flow rate of the formaldehyde-containing gas passing through the deodorizing filter medium 10 can be adjusted by controlling the area of the deodorizing filter medium 10 through which the formaldehyde-containing gas passes.

The odor reduction rate (%) of the deodorizing filter medium 10 in the aeration deodorant test was calculated by the following formula. The result is shown in Table 3.

Odor reduction rate=((formaldehyde concentration before passing through deodorizing filter medium−formaldehyde concentration after passing through deodorizing filter medium)/formaldehyde concentration before passing through deodorizing filter medium)×100.

The obtained deodorizing filter medium was cut to prepare a test specimen having a size of 10 cm×10 cm. Then, the test specimen was placed in a bag (volume: 3 liters) formed of a PVDO resin-coated PP resin film, and air containing 1500 vol ppm of formaldehyde was filled in the bag. In such a state, the test specimen was allowed to stand (25° C., 24 hours), and then the concentration of formaldehyde was measured to calculate a deodorization rate (hereinafter, referred to as a “deodorization rate after 24 hours”). The result is shown in Table 3.

### (2) Production and Evaluation of Deodorizing Paper

The deodorant-containing processing liquid (L1) was applied onto the surface of a sheet of the above-described paper by bar coating (No. 32), and then the resulting coating film was dried by heating (130° C., 3 minutes) to obtain a deodorizing paper sheet in which the deodorant composition was adhered to cellulose fibers.

The deodorizing performance (deodorization rate after 24 hours) of the obtained deodorizing paper sheet was evaluated in the same manner as in the evaluation of the deodorizing filter medium. The result is shown in Table 3.

(3) Production and Evaluation of Deodorizing Film

The deodorant-containing processing liquid (L1) was applied onto the surface of the above-described resin film by bar coating (No. 32), and then the resulting coating film was dried by heating (130° C., 3 minutes) to obtain a deodorizing film having a coating with a thickness of about 10 μm.

The deodorizing performance (deodorization rate after 24 hours) of the obtained deodorizing film was evaluated in the same manner as in the evaluation of the deodorizing filter medium. The result is shown in Table 3.

### Examples 2-2

A deodorizing filter media, deodorizing paper sheet, and deodorizing film were produced in the same manner as those in Example 2-1 except that the deodorant-containing processing liquid (L2) was used instead of the deodorant-containing processing liquid (L1) (see Table 3).

### Examples 2-3

A deodorizing filter media, deodorizing paper sheet, and deodorizing film were produced in the same manner as those in Example 2-1 except that the deodorant-containing processing liquid (L3) was used instead of the deodorant-containing processing liquid (L1) (see Table 3).

### Examples 2-4

A deodorizing filter media, deodorizing paper sheet, and deodorizing film were produced in the same manner as those in Example 2-1 except that the deodorant-containing processing liquid (L4) was used instead of the deodorant-containing processing liquid (L1) (see Table 3).

### Examples 2-5

A deodorizing filter media, deodorizing paper sheet, and deodorizing film were produced in the same manner as those in Example 2-1 except that the deodorant-containing processing liquid (L5) was used instead of the deodorant-containing processing liquid (L1) (see Table 3).

### Examples 2-6

A deodorizing filter media, deodorizing paper sheet, and deodorizing film were produced in the same manner as those in Example 2-1 except that the deodorant-containing processing liquid (L6) was used instead of the deodorant-containing processing liquid (L1) (sec Table 3).

### Examples 2-7

A deodorizing filter media, deodorizing paper sheet, and deodorizing film were produced in the same manner as those in Example 2-1 except that the deodorant-containing processing liquid (L7) was used instead of the deodorant-containing processing liquid (L1) (see Table 3).

### Examples 2-8

A deodorizing filter media, deodorizing paper sheet, and deodorizing film were produced in the same manner as those in Example 2-1 except that the deodorant-containing processing liquid (L8) was used instead of the deodorant-containing processing liquid (L1) (sec Table 3).

Production of various deodorizing products was tried using the deodorant-containing processing liquid (L9), (L10), (L11), (L12), (L13), or (L14) instead of the deodorant-containing processing liquid (L1), however, these products was not be produced due to too high a viscosity of processing liquid or aggregation of the component contained (see Table 3).

TABLE 3

| | Examples | | | | | | | | Comparative Examples | | | | | |
|---|---|---|---|---|---|---|---|---|---|---|---|---|---|---|
| | 2-1 | 2-2 | 2-3 | 2-4 | 2-5 | 2-6 | 2-7 | 2-8 | 2-1 | 2-2 | 2-3 | 2-4 | 2-5 | 2-6 |
| Deodorant-containing processing liquids | L1 | L2 | L3 | L4 | L5 | L6 | L7 | L8 | L9 | L10 | L11 | L12 | L13 | L14 |

| | | | | | | | | | |
|---|---|---|---|---|---|---|---|---|---|
| Deodorizing filter media | | | | | | | | | Deodorizing products was not |
| Impregnation amount of acid salt of aminoguanidine | 3.0 | 2.8 | 3.8 | 3.2 | 3.8 | 2.8 | 3.3 | 3.3 | be produced due to too high a |
| (g/m²) | | | | | | | | | viscosity or aggregation of |
| Odor reduction rate (%) in aeration deodorant test | 92 | 90 | 94 | 94 | 96 | 92 | 94 | 92 | the component contained. |
| Deodorization rate (%) after 24 hours | >99 | >99 | >99 | >99 | >99 | >99 | >99 | >99 | |
| Deodorizing paper sheet | | | | | | | | | |
| Impregnation amount of acid salt of aminoguanidine | 5.8 | 6.1 | 7.6 | 6.5 | 7.2 | 6.2 | 6.7 | 6.3 | |
| (g/m²) | | | | | | | | | |
| Deodorization rate (%) after 24 hours | >99 | >99 | >99 | >99 | >99 | >99 | >99 | >99 | |
| Deodorizing film | | | | | | | | | |
| Impregnation amount of acid salt of aminoguanidine | 2.9 | 3.0 | 3.5 | 3.1 | 3.5 | 2.8 | 3.1 | 3.4 | |
| (g/m²) | | | | | | | | | |
| Deodorization rate (%) after 24 hours | >99 | >99 | >99 | >99 | >99 | >99 | >99 | >99 | |

The deodorizing products (deodorizing films, deodorizing paper sheets, and deodorizing filter media) of Examples 2-1 to 2-8 were obtained using the stable deodorant-containing processing liquids (L1) to (L8) obtained in Examples 1-1 to 1-8. The deodorant-containing processing liquids (L1) to (L8) allowed the base to evenly be impregnated with the complex deodorant in high concentration, and the deodorizing products obtained in Examples 2-1 to 2-8 showed sufficiently high deodorizing performance.

According to the embodiment of the present invention, it is possible to provide a deodorant-containing processing liquid that prevents the generation of an aggregate and an increase in viscosity thereof when the deodorant-containing processing liquid contains a deodorant obtained by combining an acid salt of aminoguanidine and an inorganic carrier capable of carrying the acid salt of aminoguanidine, and the mass ratio of the acid salt of aminoguanidine to the inorganic carrier is high.

Further, according to the embodiment of the present invention, it is possible to provide a method for efficiently producing a deodorizing product having high deodorizing performance without repeatedly performing coating and drying.

Further, according to the embodiment of the present invention, it is possible to provide a deodorizing filter medium having excellent deodorizing performance against aldehyde-based gases and a deodorizing filter unit and a deodorizing apparatus which include such a deodorizing filter medium.

### INDUSTRIAL APPLICABILITY

The deodorant-containing processing liquid according to the present invention can easily be applied onto a fibrous base or the like, and is suitable for producing various deodorizing products (e.g., deodorizing filter media, deodorizing paper sheets, deodorizing films) having excellent deodorizing performance against aldehyde-based gases.

The deodorizing products obtained according to the present invention can suitably be used at sites where materials or products that generate aldehyde-based gases are used, such as building materials (e.g., plywood, laminated wood, floor boards, particle boards, heat-insulating materials), floor carpets, sound-deadening pads, cushioning materials, car seats, headrests, armrests, door trims, molded headers, sun visors, rear package trays, instrument panels, and dash insulators.

The deodorizing filter medium according to the present invention instantaneously delivers high deodorizing performance against aldehyde-based gases contained in odorous gas passing therethrough, and therefore the deodorizing filter medium according to the present invention and the deodorizing apparatus including the deodorizing filter medium according to the present invention are suitable for use in medical and care sites, toilets, sewage plants, garbage disposal plants (incinerator plants), fertilizer plants, and chemical plants.

### REFERENCE SIGNS LIST

10: Deodorizing filter media, 11: fiber, 13: deodorant (complex), 15: joined part (adhered resin and dispersant), 20: gas bag, 30: gas detector tube, 40: gas collector.

1. A deodorant-containing processing liquid, comprising an acid salt of an aminoguanidine, an inorganic carrier capable of carrying the acid salt of the aminoguanidine, a dispersant, and a dispersion medium,
wherein a content of the acid salt of the aminoguanidine is 15 parts or more by mass based on 100 parts by mass of a content of the inorganic carrier, and
the dispersant is at least one selected from the group consisting of an anionic surfactant and a nonionic surfactant.

2. The deodorant-containing processing liquid of claim 1, wherein the acid salt of the aminoguanidine is carried by the inorganic carrier.

3. The deodorant-containing processing liquid of claim 1, wherein a content ratio of the inorganic carrier is in a range of 0.1% to 50% by mass based on the deodorant-containing processing liquid.

4. A method for producing a deodorizing product, the method comprising the:
forming a coating film on a surface of a base using the deodorant-containing processing liquid of claim 1 and the base; and
drying the coating film.

5. A deodorizing filter medium, comprising a sheet-shaped base comprising a fiber and a deodorant-containing part joined to a surface of the fiber, the deodorizing filter medium having an air permeable from one surface side to other surface side,
wherein the deodorant-containing part comprises an acid salt of an aminoguanidine, an inorganic carrier carrying the acid salt of the aminoguanidine, an adhesive resin, and a surfactant,
a content of the acid salt of the aminoguanidine is 15 parts or more by mass based on 100 parts by mass of a content of the inorganic carrier, and
the surfactant is at least one selected from the group consisting of an anionic surfactant and a nonionic surfactant.

6. The deodorizing filter medium of claim 5, wherein a content ratio of the acid salt of the aminoguanidine is in a range of 1% to 50% by mass based on the base.

7. The deodorizing filter medium of claim 5, which has an air permeability in a range of 10 to 500 cm³/(cm²·s).

8. A deodorizing filter unit, comprising the deodorizing filter medium of claim 5 and a carrying member that carries the deodorizing filter medium.

9. A deodorizing apparatus, comprising the deodorizing filter medium of claim 5.
